(19) United States
(12) Patent Application Publication
(10) Pub. No.: US 2013/0225974 A1
(43) Pub. Date: Aug. 29, 2013

(54) MAGNETIC RESONANCE IMAGING SYSTEM AND RADIOTHERAPY APPARATUS WITH AN ADJUSTABLE AXIS OF ROTATION

(71) Applicant: Johan Samuel Van Den Brink, Meteren (NL)

(72) Inventor: Johan Samuel Van Den Brink, Meteren (NL)

(73) Assignee: KONINKLIJKE PHILIPS ELECTRONICS N.V., EINDHOVEN (NL)

(21) Appl. No.: 13/883,917

(22) PCT Filed: Oct. 31, 2011
(86) PCT No.: PCT/IB11/54818

§ 371 Date: May 7, 2013

(30) Foreign Application Priority Data
Nov. 9, 2010 (EP) 10190485.2

(51) Int. Cl. A61N 5/10; A61B 5/055

(52) U.S. Cl. CPC A61N 5/1039; A61B 5/055; A61N 5/1081; A61N 5/1048
USPC 600/411

(57) ABSTRACT

A therapeutic apparatus (100) comprising: a radio therapy apparatus (102) for treating a target zone (146) of a subject (144), wherein the radio therapy apparatus comprises a radio therapy source (110) for generating electromagnetic radiation (114), wherein the radio therapy apparatus is adapted for rotating the radio therapy source about a rotational point (116); a mechanical actuator (104) for supporting the radio therapy apparatus and for moving the position and/or orientation of the rotational point; and a magnetic resonance imaging system (106) for acquiring magnetic resonance data (170) from an imaging zone (138), wherein the target zone is within the imaging zone, wherein the magnetic resonance imaging system comprises a magnet (122) for generating a magnetic field within the imaging zone, wherein the radio therapy source is adapted for rotating at least partially about the magnet.

### TECHNICAL FIELD

The invention relates to apparatuses for treating a target zone of a subject with radiotherapy, in particular the invention relates to radiotherapy apparatuses guided by magnetic resonance imaging.

### BACKGROUND OF THE INVENTION

Integration of MR and Linear Accelerators (LINAC) opens new horizons in Radiotherapy by improved lesion targeting, especially for moving organs. In a practical implementation proposal, the LINAC rotates around the patient to hit the gross target volume (GTV) and clinical target volume (CTV) from multiple angles while minimizing the radiation exposure for surrounding tissues. In routine practice of Radiotherapy (RT), the patient is positioned relative to the stationary center of the rotating arc carrying the RT source. Positioning implies both height and lateral adjustment of the patient table. This positioning is required to optimize the dose in the lesion beyond variation that can be obtained by applying RT rays from different angles.

U.S. Pat. No. 6,198,957 discloses a radiotherapy machine for beam treating a region of a subject combined with a magnetic resonance imaging system. The beam and the excitation coil assembly of the imaging system are arranged so that the beam is not incident on the coil assembly.

### SUMMARY OF THE INVENTION

The invention provides for a therapeutic apparatus, a computer program product, and a computer-implemented method in the independent claims. Embodiments are given in the dependent claims.

While performing radiotherapy the radiotherapy source is typically moved to a variety of positions while irradiating a target zone. This is done to minimize the exposure portions of a subject which do not include the target zone to the effects of the radiation. Typically, this is done by rotating the radiotherapy source about an axis of rotation.

A difficulty encountered in guiding radiotherapy treatments using magnetic resonance (MR) imaging is the limited space in magnets that are useful for clinical imaging, such as cylindrical superconducting magnets. For such magnets there is simply is not sufficient space in a magnet to position the target zone relative to the rotational axis of the radiotherapy source.

Embodiments of the invention address this problem by mounting a radiotherapy apparatus on a mechanical actuator that can move the rotational point and or changing the orientation of the rotational point of the radiotherapy source. The radiotherapy source rotates about a rotational axis within a rotational plane. The intersection of the rotational axis and the rotational plane is the rotational point. The direction of the rotational axis provides or defines the orientation for the rotational point. The rotational axis does not have a preferential direction, so the direction of the orientation of the rotational point is chosen. In other words, the mechanical actuator can move the location of the rotational point relative to the isocenter of the magnet, and/or can change the orientation of the rotational axis relative to a symmetry axis of the magnet.

The radiotherapy source may be designed such that objects within a predetermined distance may be irradiated by the radiotherapy source. In some embodiments, the radiotherapy source may be equipped with an adjustable beam collimator, such as a multi leaf collimator, to control the path of the radiation beam.

When integrating MR and a LINAC, the source is placed outside the magnet.

As discussed above, the patient space in a cylindrical magnet is quite compromised, and moving the patient inside the magnet relative to the LINAC source is very difficult. Positioning along the foot-head axis is possible, but with standard mechatronics of MR patient supports not within more than 15 mm accuracy. The readout of the location is much better, and will be supplied to the RT planning system for accurate beam steering. Where positioning the patient in Left-Right direction is compromised due to the space in the magnet bore, adjustment of patient location in Anterior-Posterior direction is virtually impossible. Thus, dose optimization would be severely compromised relative to state of the art RT solutions.

Since the magnet frame of reference is fixed, and the patient cannot be moved relative to the RT setup, the only solution is to move the RT relative to the magnet isocenter line. Such a workflow does not significantly interfere with RT requirements: the LINAC is rotated around the patient (and magnet), and stopped at pre-calculated angles to apply the required radiation dose. This is a relatively slow process, which can easily be extended by a movement of the center line of the LINAC relative to the magnet center line. For illustration see next page.

This additional degree of freedom may be included in the RT planning software for optimal results: calculate the dose per rotated and shifted position. The Linear Accelerator is placed in a zero-field envelope outside the magnet. For optimal design and maneuverability of the LINAC in AP and LR direction, the zero-field envelope must be as wide as possible, and wider than for a stationary position of the LINAC. Typical dimension would be 15 cm for a stationary LINAC, and up to 30 cm for the moving source.

A computer-readable storage medium as used herein encompasses any tangible storage medium which may store instructions which are executable by a processor of a computing device. The computer-readable storage medium may be referred to as a computer-readable non-transitory storage medium. The computer-readable storage medium may also be referred to as a tangible computer readable medium. In some embodiments, a computer-readable storage medium may also be able to store data which is able to be accessed by the processor of the computing device. Examples of computer-readable storage media include, but are not limited to: a floppy disk, a magnetic hard disk drive, a solid state hard disk, flash memory, a USB thumb drive, Random Access Memory (RAM) memory, Read Only Memory (ROM) memory, an optical disk, a magneto-optical disk, and the register file of the processor. Examples of optical disks include Compact Disks (CD) and Digital Versatile Disks (DVD), for example CD-ROM, CD-RW, CD-R, DVD-ROM, DVD-RW, or DVD-R disks. The term computer readable-storage medium also refers to various types of recording media capable of being accessed by the computer device via a network or communication link. For example a data may be retrieved over a modem, over the internet, or over a local area network.

Computer memory is an example of a computer-readable storage medium. Computer memory is any memory which is directly accessible to a processor. Examples of computer memory include, but are not limited to: RAM memory, registers, and register files.

Computer storage is an example of a computer-readable storage medium. Computer storage is any non-volatile computer-readable storage medium. Examples of computer storage include, but are not limited to: a hard disk drive, a USB thumb drive, a floppy drive, a smart card, a DVD, a CD-ROM, and a solid state hard drive. In some embodiments computer storage may also be computer memory or vice versa.

A computing device as used herein refers to any device comprising a processor. A processor is an electronic component which is able to execute a program or machine executable instruction. References to the computing device comprising “a processor” should be interpreted as possibly containing more than one processor. The term computing device should also be interpreted to possibly refer to a collection or network of computing devices each comprising a processor. Many programs have their instructions performed by multiple processors that may be within the same computing device or which may even distributed across multiple computing device.

A user interface as used herein encompasses an interface which allows a user or operator to interact with a computer or computer system. A user interface may provide information or data to the operator and/or receive information or data from the operator. The display of data or information on a display or a graphical user interface is an example of providing information to an operator. The receiving of data through a keyboard, mouse, trackball, touchpad, pointing stick, graphics tablet, joystick, gamepad, webcam, headset, gear sticks, steering wheel, pedals, wired glove, dance pad, remote control, and accelerometer are all examples of receiving information or data from an operator. Magnetic Resonance (MR) data is defined herein as being the recorded measurements of radio frequency signals emitted by atomic spins by the antenna of a Magnetic resonance apparatus during a magnetic resonance imaging scan. A Magnetic Resonance Imaging (MRI) image is defined herein as being the reconstructed two or three dimensional visualization of anatomic data contained within the magnetic resonance data. This visualization can be performed using a computer.

In one aspect, the invention provides for a therapeutic apparatus comprising a radiotherapy apparatus for treating a target zone of a subject. As used herein a radiotherapy apparatus encompasses an apparatus which generates high energy electromagnetic radiation for performing radiotherapy. A radiotherapy apparatus may for example be, but is not limited to: an x-ray system, a LINAC system and a radioisotope therapy apparatus. A radioisotope therapy apparatus uses a radioisotope to generate the high energy electromagnetic radiation. In some instances the high energy electromagnetic radiation may be ionizing electromagnetic radiation. That is to say the energy of the photons is high enough to break chemical bonds or cause cell necrosis.

The radiotherapy apparatus comprises a radiotherapy source for generating electromagnetic radiation. The electromagnetic radiation is used to treat target zone. The radiotherapy apparatus is adapted for rotating the radiotherapy source about a rotational point. The therapeutic apparatus further comprises a mechanical actuator for supporting the radiotherapy apparatus and for moving the position and/or orientation of the rotational point. In other words the mechanical actuator is able to support and move the radiotherapy apparatus. In some embodiments the mechanical actuator may move the radiotherapy apparatus within a plane. For example relative to the rotational axis the mechanical actuator may move the radiotherapy apparatus in the two directions perpendicular to the rotational axis. In other embodiments the mechanical actuator may rotate the rotational axis in order to move it. In other embodiments the mechanical actuator may tilt the entire radiotherapy apparatus.

In other embodiments the position of the rotational axis is able to be adjusted three-dimensionally. Being able to adjust the position of the rotational axis may be advantageous when conventional subject supports with positioning systems are used. The positioning systems may not have fine enough control to accurately position the subject correctly to allow the radio therapy apparatus to treat or irradiate the target zone. Adjusting the position and or orientation of the rotational point enables proper irradiation of the target zone.

The therapeutic apparatus further comprises a magnetic resonance imaging system for acquiring magnetic resonance data from an imaging zone. The target zone is within the imaging zone. This is beneficial because the magnetic resonance data is able to acquire anatomical data of the subject in the vicinity of the target zone. The magnetic resonance imaging system may therefore be used for several different purposes. For instance the magnetic resonance imaging system may be used to guide the radiotherapy apparatus during treatment of the target zone. In some instances the magnetic resonance imaging system may also be used for taking pre- and post-magnetic resonance data to assess the effectiveness of the treatment of the target zone. The magnetic resonance imaging system comprises a magnet for generating a magnetic field within the imaging zone. The radiotherapy source is adapted for rotating at least partially about the magnet.

Several different types of magnets may be used for implementation of embodiments of the invention. Cylindrical superconducting magnets with a bore for receiving the subject are typically used for magnetic resonance imaging systems. The magnetic resonance imaging system can be designed such that magnetic radiation from the radiotherapy source may pass through the walls of the magnet and then through the subject. Other types of magnets may also be used. In particular the so called open magnets for magnetic resonance imaging may also be used. Open magnetic resonance imaging magnets have two sections of magnet with a space between the two sections. The subject goes between the two sections of magnet. For this type of magnet the radiotherapy apparatus may still be placed such that it rotates at least partially about the magnet. Rotating about the magnet may also be interpreted as rotating around and/or outside the magnet.

The magnets for magnetic resonance imaging systems are typically expensive. As the size of magnet increases, the cost of the magnet increases greatly. For this reason when magnetic resonance imaging magnets are designed the bore of the magnet is typically just large enough to receive the subject. This may be a disadvantage when treating the target zone of the subject with the electromagnetic radiation. Embodiments of the invention may have the advantage that because the position and/or orientation of the rotational point can be controlled by the mechanical actuator and the rotation of the radiotherapy source can allow positioning of the radiotherapy source such that the target zone of the subject can be reached for multiple rotational positions of the radiotherapy source. This allows the treatment of the target zone of the subject from multiple angles. This has the benefit that it may reduce the amount of ionizing radiation that reaches the subject's anatomy outside the target zone. In simpler terms embodiments of this therapeutic apparatus may have the advantage of allowing treatment to target zones of the subject which do not lie on a primary axis of the magnet.

Since the radiotherapy apparatus and the magnetic resonance imaging system have a mutual axis of symmetry, the radiotherapy source may have only limited capabilities to reach a target zone of the subject which is not on this axis.

When performing radiotherapy, the subject is typically placed on a subject support with six degrees of freedom. This allows precise positioning of the target zone such that it can be effectively treated by the radiotherapy source. The use of the magnetic resonance imaging magnet severely restricts how a subject can be moved. The addition of a mechanical actuator which allows the positioning of the rotational point and/or control of the orientation of the rotational point of the radiotherapy apparatus may allow for more effective and precise treatment of the subject.

In another embodiment the therapeutic apparatus further comprises a processor for controlling the therapeutic apparatus. The processor may be considered to be equivalent with a computer system for controlling the therapeutic apparatus and also as a control system for controlling the therapeutic apparatus. The therapeutic apparatus further comprises a memory containing machine executable instructions for execution by the processor. As used herein a processor is understood to encompass a collection of processors in a single machine and/or processors distributed amongst multiple machines. For instance a collection of computers which are networked together may function and perform the task of controlling the therapeutic apparatus.

Execution of the instructions causes the processor to acquire the magnetic resonance data using the magnetic resonance imaging system. That is to say the instructions cause the processor to control the magnetic resonance imaging system such that magnetic resonance data is acquired. Execution of the instructions further causes the processor to reconstruct a magnetic resonance image from a magnetic resonance data. As used herein a magnetic resonance image may refer to multiple images such as that which are currently referred to as slices. The magnetic resonance data may have been primarily acquired from a particular volume. When reconstructed multiple images or slices may be made to construct the magnetic resonance image. It is understood that reference to a magnetic resonance image may also refer to multiple images.

Execution of the instructions further causes the processor to register a location of the target zone in the magnetic resonance image. Using well known image recognition techniques or registration techniques anatomical landmarks may be located within the magnetic resonance image and used to register the location of the target zone in the magnetic resonance image. Execution of the instructions further cause the processor to generate actuator control signals in accordance with the location of the target zone. Actuator control signals cause the mechanical actuator to move the position and/or orientation of the rotational point. Execution of the instructions further cause the processor to generate radiotherapy control signals in accordance with the location of the target zone. The radiotherapy control signals causes the radiotherapy apparatus to irradiate the target zone and cause the radiotherapy apparatus to control rotation of the radiotherapy source about the rotational axis.

In some embodiments the radiotherapy control signals may be identical with the actuator control signals. In some embodiments there may be control signals which comprise both the actuator control signals and the radiotherapy control signals. The radiotherapy control signals contain commands which control both the movement of the radiotherapy source and the operation of the radiotherapy source. Execution of the instructions further cause the processor to send the actuator control signals to the mechanical actuator. Execution of the instructions further cause the processor to send the radiotherapy control signals to the radiotherapy apparatus. The actuator control signals and the radiotherapy control signals may be sent for example by a connection over a computer network or interface.

In another embodiment execution of the instructions cause the processor to generate actuator control signals that cause the mechanical actuator to move such that the rotational point is within a predetermined distance to the target zone. This embodiment is particularly advantageous because if the mechanical actuator positions and/or orientates the rotational point in such a way the radiotherapy source may always be positioned such that the electromagnetic radiation it generates will pass through the target zone.

In another embodiment electromagnetic radiation generated by the radiotherapy source passes through the rotational point.

In another embodiment execution of the instructions further causes the apparatus to register a location of a critical anatomy zone in the magnetic resonance image. The registration of the critical anatomy zone may for instance be achieved using known image recognition and registration techniques. Actuator control signals are generated in accordance with the location of the target zone and the critical anatomy zone such that the radiation dose to the critical anatomy zone is minimized and that the radiation dose to the target zone is maximized. This embodiment may be beneficial in a situation where it is beneficial to the subject if the critical anatomy zone is not irradiated with the electromagnetic radiation. For instance the critical anatomy zone may outline a position of a critical organ.

In another embodiment the therapeutic apparatus further comprises a subject support control interface for controlling a subject support for positioning the subject. The subject support control interface may take different forms in different embodiments. For instance the subject support control interface may be a component of a computer system which is connected to the processor. In other instances the subject support control interface may be an interface which is built into the subject support. The subject support may also be able to control different degrees of freedom of the positioning subject depending upon different embodiments. In one embodiment the subject support may only be able to position the subject moving along a single axis. For instance when the subject is placed into a magnetic resonance imaging magnet and there is hardly enough clearance for the subject the subject support may be designed or operated such that the subject is only moved along the axis of the magnet.

Execution of the instructions further causes the processor to generate subject support control signals. Execution of the instructions further cause the processor to send the subject support control signals to the subject support using the subject support interface. The subject support control signals are generated in accordance with the radiotherapy control signals and the location of the target zone. The subject support control signals are signals or commands which cause the subject support to change the position of the subject. In some embodiments they also may change the orientation of the subject. The subject support control signals are generated in conjunction with the radiotherapy control signals, the location of the target zone, and/or the actuator control signals so that the target zone is irradiated precisely by the radiotherapy source.

In another embodiment the therapeutic apparatus comprises the subject support for positioning the subject.

In another embodiment execution of the instructions further cause the processor to repeatedly acquire the magnetic resonance data, reconstruct the magnetic resonance image, and register the location of the target zone during irradiation of the target zone. Execution of the instructions further causes the processor to repeatedly generate and send updated radiotherapy control signals. The updated radiotherapy control signals compensate for motion of the subject between subsequent acquisitions of the magnetic resonance data. Execution of the instructions further cause the processor to repeatedly send the updated radiotherapy control signals to the radiotherapy source during irradiation of the target zone. This embodiment is particularly advantageous because the magnetic resonance imaging system is used for guiding the treatment of the target zone by the radiotherapy apparatus. The magnetic resonance imaging system is used to register changes in the anatomy due to movement of the subject and to create control signals or commands which compensate for this.

In some embodiments the actuator control signals and the subject support control signals are also repeatedly generated and repeatedly sent in the same way that the radiotherapy control signals are.

In another embodiment the radiotherapy apparatus comprises an adjustable beam collimator. The updated radiotherapy control signals comprise commands for controlling the beam collimator. This embodiment is particularly advantageous because it may be difficult to rapidly move a subject support, the radiotherapy source, or the mechanical actuator to compensate for motion of the subject. The adjustable beam collimator however may be very rapidly adjusted using small actuators or mechanisms. The beam collimator may for example be, but is not limited to, a multi leaf collimator.

In another embodiment the radiotherapy source is adapted for generating a radiator beam with a beam path. The radiotherapy rotates the radiotherapy source within a rotational plane. The radiotherapy apparatus further comprises a tilt apparatus adapted for tilting the beam path relative to the rotational plane. This embodiment is advantageous because by tilting the radiotherapy source it is possible for the beam path to reach the target zone in a way which avoids portions of the subject which are not part of the target zone.

In another embodiment execution of the instructions further causes the processor to generate tilt apparatus control signals in accordance with the location of the target zone. The tilt apparatus control signals cause the tilt apparatus to tilt the beam path relative to the rotational plane. The radiotherapy control signals comprise the tilt apparatus control signals.

In another embodiment the radiotherapy source is a LINAC for generating x-ray or gamma radiation. The magnet is adapted for generating a low magnetic field zone which encircles the magnet. The radiotherapy apparatus is adapted such that the radiotherapy source rotates about the magnet within the low magnetic field zone. The magnetic field strength within the low magnetic field zone is below an operational threshold of the LINAC source. The operational threshold defines a magnetic field strength which prevents the LINAC source from functioning properly. In modern cylindrical bore magnetic resonance imaging magnets there are typically several compensation coils. The compensation coils generate a magnetic field which is opposed to coils used to generate the main magnetic field. This results in an area outside of the cylindrical magnet approximately in the mid-plane which is doughnut-shaped and has a low magnetic field. The low magnetic field zone may be this doughnut-shaped zone surrounding the cylindrical magnet with compensation coils.

In another embodiment the operational threshold is below 5 mT, preferably below 10 mT.

In another embodiment the radiotherapy source is a LINAC x-ray source or a LINAC gamma ray source.

In another embodiment the radiotherapy source is an x-ray tube.

In another embodiment the radiotherapy source is a radioisotope gamma radiation source. A radioisotope gamma radiation source uses a radioisotope to produce gamma radiation.

In another embodiment the mechanical actuator comprises a hydraulic system. The use of a hydraulic system may be beneficial because hydraulic systems can be used to lift very heavy objects. In addition for the hydraulic system can be located away from the magnetic resonance imaging system. This saves valuable space in the examination room and also the machinery used to lift or move the mechanical actuator is away from the magnetic resonance imaging system and therefore can be designed to function without concern to the high magnetic field generated by the magnetic resonance imaging magnet.

In another aspect the invention provides for a computer program product comprising machine executable instructions for execution by a processor of a therapeutic apparatus. The therapeutic apparatus comprises a radiotherapy apparatus for treating a target zone of a subject. The radiotherapy apparatus comprises a radiotherapy source for generating electromagnetic radiation. The radiotherapy apparatus is adapted for rotating the radiotherapy source about a rotational point. The therapeutic apparatus further comprises a mechanical actuator for supporting the radiotherapy apparatus and for moving the position and/or orientation of the rotational point. The therapeutic apparatus further comprises a magnetic resonance imaging system for acquiring magnetic resonance data from an imaging zone. The target zone is within the imaging zone. The magnetic resonance imaging system comprises a magnet for generating a magnetic field within the imaging zone. The radiotherapy source is adapted for rotating at least partially about the magnet. Execution of the instructions causes the processor to acquire the magnetic resonance data using the magnetic resonance imaging system. Execution of the instructions further causes the processor to reconstruct a magnetic resonance image from the magnetic resonance data.

Execution of the instructions further causes the processor to register a location of the target zone in the magnetic resonance image. Execution of the instructions further causes the processor to generate actuator control signals in accordance with the location of the target zone. The actuator control signals cause the mechanical actuator to move the position and/or orientation of the rotational point. Execution of the instructions further causes the processor to generate radiotherapy control signals in accordance with the location of the target zone. The radiotherapy control signals causes the radiotherapy apparatus to irradiate the target zone and cause the radiotherapy apparatus to control rotation of the radiotherapy source about the rotational axis. Execution of the instructions further causes the processor to send the actuator control signals to the mechanical actuator. Execution of the instructions further causes the processor to send the radiotherapy control signals to the radiotherapy apparatus.

The computer program product may for instance be stored on a computer-readable storage medium.

In another aspect the invention provides for a computer-implemented method of controlling a therapeutic apparatus. The invention also provides for a method of controlling a therapeutic apparatus which corresponds to the computer-implemented method. The therapeutic apparatus comprises a radiotherapy apparatus for treating a target zone of the subject. The radiotherapy apparatus comprises a radiotherapy source for generating electromagnetic radiation. The radiotherapy apparatus is adapted for rotating the radiotherapy source about a rotational point. The therapeutic apparatus further comprises a mechanical actuator for supporting the radiotherapy apparatus and for moving the position and/or orientation of the rotational point. The therapeutic apparatus further comprises a magnetic resonance imaging system for acquiring magnetic resonance data from an imaging zone. The target zone is within the imaging zone. The magnetic resonance imaging system comprises a magnet for generating the magnetic field within the imaging zone. The radiotherapy source is adapted for rotating at least partially about the magnet.

The method comprises the step of acquiring magnetic resonance data using a magnetic resonance imaging system. The method further comprises the step of reconstructing the magnetic resonance image from the magnetic resonance data. The method further comprises the step of registering a location of the target zone in the magnetic resonance image. The method further comprises the step of generating actuator control signals in accordance with the location of the target zone. The actuator control signals cause the mechanical actuator to move the position of the rotational access. The method further comprises the step of generating radiotherapy control signals in accordance with the location of the target zone. The radiotherapy control signals causes the radiotherapy apparatus to irradiate the target zone and cause the radiotherapy apparatus to control rotation of the radiotherapy source about the rotational point. The method further comprises the step of sending the actuator control signals to the mechanical actuator. The method further comprises the step of sending the radiotherapy control signals to the radiotherapy apparatus.

### BRIEF DESCRIPTION OF THE DRAWINGS

In the following preferred embodiments of the invention will be described, by way of example only, and with reference to the drawings in which:

FIG. 1 shows a cross-sectional and functional view of a therapeutic apparatus according to an embodiment of the invention;

FIG. 2 shows a further cross-sectional view perpendicular to the rotational axis of the therapeutic apparatus shown in FIG. 1;

FIG. 3 shows a further cross-sectional view perpendicular to the rotational axis of the therapeutic apparatus shown in FIG. 1;

FIG. 4 shows a further cross-sectional view perpendicular to the rotational axis of the therapeutic apparatus shown in FIG. 1;

FIG. 5 shows a further cross-sectional view perpendicular to the rotational axis of the therapeutic apparatus shown in FIG. 1;

FIG. 6 shows a flow diagram which illustrates a method according to an embodiment of the invention; and

FIG. 7 shows a flow diagram which illustrates a method according to a further embodiment of the invention.

### DETAILED DESCRIPTION OF THE EMBODIMENTS

Like numbered elements in these figures are either equivalent elements or perform the same function. Elements which have been discussed previously will not necessarily be discussed in later figures if the function is equivalent.

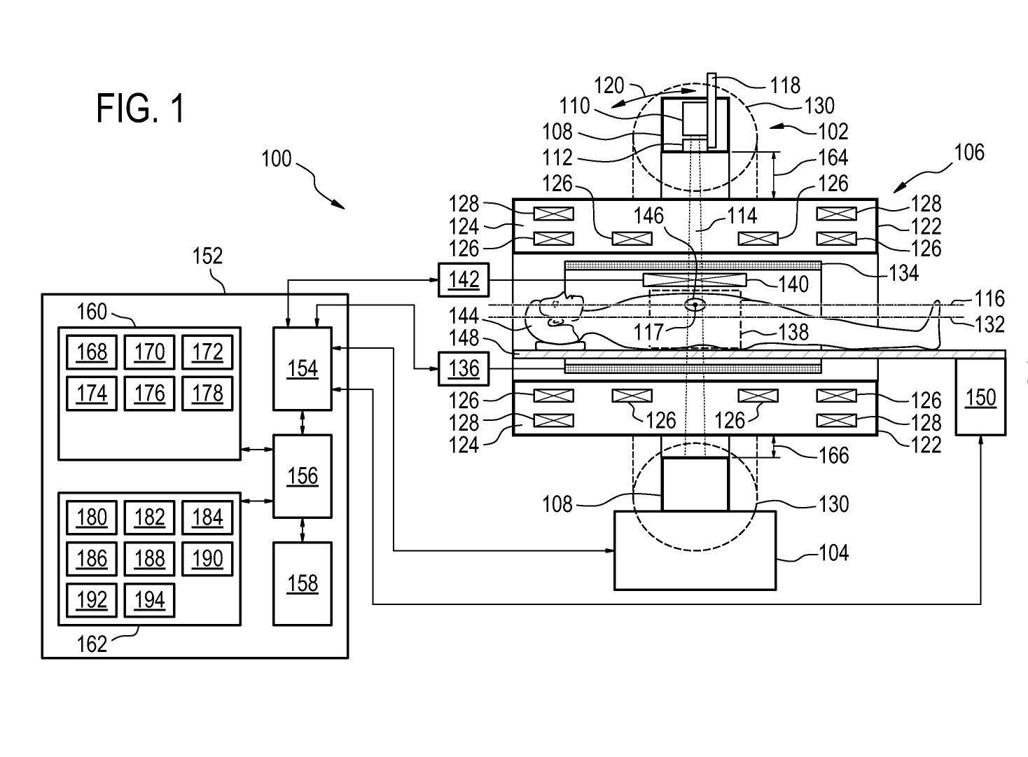

FIG. 1 shows a cross-sectional and functional view of a therapeutic apparatus 100 according to an embodiment of the invention. The therapeutic apparatus 100 is shown as comprising a radiotherapy apparatus 102, a mechanical actuator 104 and a magnetic resonance imaging system 106. The radiotherapy apparatus 102 comprises a ring mechanism 108. The ring mechanism 108 supports a radiotherapy source 110. The radiotherapy source 110 is representative and may be a LINAC x-ray source, an x-ray 2 and a radioisotope gamma radiation source. Adjacent to the radiotherapy source 110 is a beam collimator 112 for collimating a radiation beam 114 that is generated by the radiotherapy source 110. The ring mechanism 108 is also adapted for rotating the radiotherapy source 100 and the beam collimator 112 about a rotational point 117 of the radiotherapy apparatus 102. A rotational axis 116 passes through the rotational point 116.

There is also a tilt apparatus 118 in the ring mechanism 108 that is adapted for tilting the radiotherapy source 110 and the beam collimator 112. The tilt apparatus 118 is adapted for tilting the angle of the radiation beam 114 relative to a plane which is perpendicular to the rotational axis 116. The magnetic resonance imaging system 106 is shown as comprising a magnet 122. The ring mechanism 108 is ring-shaped and surrounds the magnet 122. The magnet 122 shown in FIG. 1 is a cylindrical type superconducting magnet. However, other magnets are also applicable for embodiments of the invention. The magnet 122 has a supercooled cryostat 124. Inside the cryostat 124 there is a collection of superconducting coils 126. There are also compensation coils 128 whose current opposes the direction of current in the superconducting coils 126. This creates a low magnetic field zone 130 that circles or encompasses the magnet 122. The cylindrical magnet 122 is shown as having an axis 132 of symmetry.

Within the bore of the magnet there is a magnetic field gradient coil 134 which is used for acquisition of magnetic resonance data to spatially encode objects within an imaging zone 138 of the magnet 122. The magnetic field gradient coil 134 is connected to a magnetic field gradient coil power supply 136. The magnetic field gradient coil 134 is intended to be representative. Typically magnetic field gradient coils contain three separate sets of coils for spatially encoding in three orthogonal spatial directions. The imaging zone 138 is located in the centre of the magnet 122.

Adjacent to the imaging zone 138 is a radio frequency coil 140 for manipulating the orientations of magnetic spins within the imaging zone 138 and for receiving radio transmissions from spins also within the imaging zone 138. The radio frequency coil 140 is connected to a radio frequency transceiver 142. The radio frequency coil 140 and radio frequency transceiver 142 may be replaced by separate transmit and receive coils and a separate transmitter and receiver. It is understood that the radio frequency coil 140 and the radio frequency transceiver 142 are simply representative.

Within the center of the magnet is also located a subject 144. The subject 144 has a target zone 146 and is shown as reposing on a subject support 148. The subject support 148 has a mechanical positioning system 150. The mechanical positioning system is adapted for positioning the subject 144 within the magnet 122. Depending upon the space available inside of the magnet the subject support 148 may be adapted for moving the subject in different directions. In this embodiment there is not much additional space for the subject 144. It is possible in one embodiment the mechanical positioning system 150 only moves the subject support in a direction perpendicular to the magnet axis 132. If there is more space available inside the magnet the mechanical positioning system 150 may have more degrees of freedom. For instance the mechanical positioning system 150 may position the subject support 148 with six degrees of freedom. The radio frequency transceiver 142, the magnetic field gradient coil power supply 136, the mechanical actuator 104, and the mechanical positioning system 150 are all shown as being connected to a hardware interface 154 of a computer system 152. The computer system 152 uses a processor 156 to control the therapeutic apparatus 100.

The computer system 152 shown in FIG. 1 is representative. Multiple processors and computer systems may be used to represent the functionality illustrated by this single computer system 152. The computer system 152 comprises the hardware interface 154 which allows the processor 156 to send and receive messages to components of the therapeutic apparatus 100. The processor 156 is also connected to a user interface 158, computer storage 160, and computer memory 162. The radiotherapy apparatus 102 is not shown as being connected to the hardware interface 154. In some embodiments the radiotherapy apparatus 102 may be connected to the hardware interface 154. In this embodiment the radiotherapy apparatus 102 communicates with the computer system 152 via the mechanical actuator 104.

For the example shown in FIG. 1, the rotational axis 116 of the radiotherapy apparatus is not coaxial with the magnet axis 132. The rotational point 117 is shown as being off center from the magnet axis 132. It can be seen that the target zone 146 is off-center and away from the magnet axis 132. The radiotherapy apparatus 102 has been moved by mechanical actuator 104 such that the rotational point 117 of the radiotherapy apparatus is within the target zone 146. It can be seen that the ring mechanism 108 has been moved relative to the magnet 122. The arrow 164 indicates a top distance between the inside of the ring mechanism 108 and arrow 166 indicates a distance between the magnet 122 and the bottom inside of the ring mechanism 108. The distance 166 is shorter than the distance 164 and it can be seen that the rotational point 117 is above the magnet axis 132. In this embodiment the radiation beam 114 passes through the rotational point 117. Placing the rotational point 117 at the center of the target zone 146 allows the target zone to be treated continuously when the radiation beam 114 is created by the radiotherapy source 110 and is rotated by the ring mechanism 108.

Computer storage 160 is shown as containing a treatment plan 168. The treatment plan 168 contains instructions or a plan for treating the target zone 146. The treatment plan 168 may contain details of the subject anatomy 144 in relation to the target zone 146. The computer storage 160 is further shown as containing magnetic resonance data 170 that has been acquired by the magnetic resonance imaging system 106. The computer storage 160 is shown as further containing a magnetic resonance image 172 that has been reconstructed from the magnetic resonance data. The computer storage 160 is shown as further containing coordinates 174 of the target zone 146 which have been determined by registering the magnetic resonance image 172. The computer storage 160 is further shown as containing actuator control signals 176. The computer storage 160 is shown as further containing radiotherapy control signals 178. The actuator control signals 176 contains instructions which can be used by the actuator 104 for controlling movement and/or orientation of the rotational axis 117 relative to the magnet axis 132.

The computer memory 162 contains machine executable instructions 180, 182, 184, 186, 188, 190, 192, 194 for operation by the processor 156. The computer memory 162 is shown as containing a therapeutic apparatus control module 180. The therapeutic apparatus control module 180 contains machine executable instructions which allow the processor 156 to control the overall functioning of the therapeutic apparatus 100. The computer memory 162 is shown as further containing a radiotherapy apparatus control module 182. The radiotherapy apparatus control module 182 contains machine executable instructions which allow the processor 156 to control the functioning of the radiotherapy apparatus 102. The computer memory 162 is shown as further containing mechanical actuator control module 184. The mechanical actuator control module 184 contains machine executable code which allows the processor 156 to communicate with the mechanical actuator 104 for controlling its function and operation.

The computer memory 162 is shown as further containing a magnetic resonance imaging control module 186. The magnetic resonance imaging control module contains machine executable code which allows the processor 156 to control the functioning and operation of the magnetic resonance imaging system. The computer memory 162 is shown as further containing an image reconstruction module 188. The image reconstruction module 188 contains machine executable code which is used by the processor 156 to transform the magnetic resonance data 170 into the magnetic resonance image 172. The computer memory 162 is further shown as containing an image registration module 190. The image registration module 190 is able to perform a registration on the magnetic resonance image 172 to determine coordinates 174 of the target zone 146. The image registration module 190 may in some embodiments use the treatment plan 168 for identification and registration of the coordinates 174 of the target zone 146.

The computer memory 162 is shown as further containing an actuator control signal generation module 192. The actuator control signal generation module 192 uses the coordinates of the target zone 174 and some embodiments the treatment plan 168 to generate the actuator control signals 176. The computer memory 162 is shown as further containing radiotherapy control signal generation module 194. The radiotherapy control signal generation module 194 contains computer executable code which the processor 156 uses to generate the radiotherapy control signals 178. The radiotherapy control signals 178 may be generated in conjunction with the actuator control signals 176, the coordinates of the target zone 174, and in some embodiments the treatment plan 168.

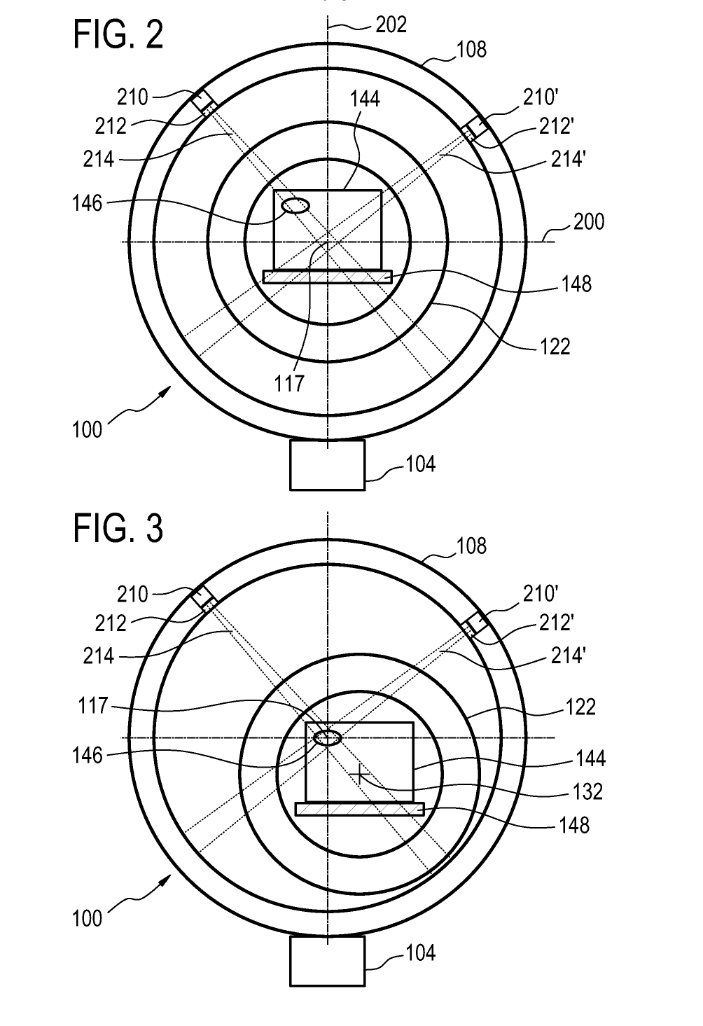

FIG. 2 shows a cross-sectional view of the therapeutic apparatus 100 shown in FIG. 1. The cross-sectional view in FIG. 2 is in the plane perpendicular to the rotational axis of the radiotherapy apparatus. In this Fig. the ring mechanism 108 is centered such that the rotational point 117 is centered on the axis of the magnet 122. Within the magnet there is the subject 144 on subject support 148. The target zone 146 is located off-center and away from the axes of both the ring mechanism 108 and the magnet 122. The x-axis 200 and the y-axis 202 lie in the rotational plane. The x 200 and y 202 axes span the rotational plane of the radiotherapy source. The radiotherapy source 210 is shown in two locations for its rotation about the rotational point 117. In a first position the radiotherapy source 210, the beam collimator 212 and the radiation beam 214 are shown such that the radiation beam 214 passes through the target zone 146. The radiotherapy source 210′, the beam collimator and the beam collimator 212′ are rotated to a second position. The radiation beam 214′ is shown as passing through the rotational point 117 but not through the target zone 146. FIG. 2 illustrates the difficulty of treating the target zone 146 without using the invention. The subject 144 is constrained to the inside of the magnet 122 and it would not be possible to move the subject support 148 such that the target zone 146 is located at the rotational point 117.

FIG. 3 shows the same cross-sectional view of the therapeutic apparatus 100 as was shown in FIG. 2. However, the rotational point 117 has been shifted to the center of the target zone 146. The rotational axis of the radiotherapy apparatus and the axis 132 of the magnet 122 are no longer coaxial. However, it can be seen in this Fig. that the radiation beams 214 and 214′ both pass through the target zone 146.

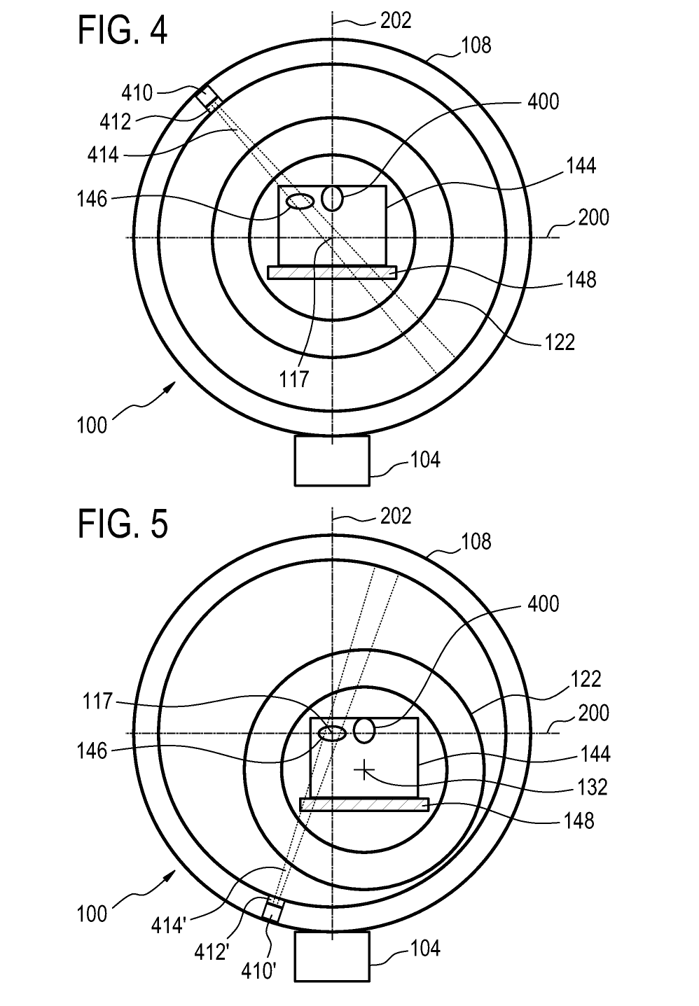

FIGS. 4 and 5 illustrate how an embodiment of the invention can be used to avoid irradiating a critical anatomy zone 400. The cross-sectional view is the same as was shown in FIGS. 2 and 3. In FIG. 4 the rotational point 117 is aligned with the axis of the magnet. The radiotherapy source 410 and the beam collimator 412 are rotated by the ring mechanism 108 such that the radiation beam 414 passes through the target zone 146 of the subject 144. Adjacent to the target zone 146 is a critical anatomy zone 400. It is desirable to avoid irradiating the critical anatomy zone 400. If the rotational point 117 is placed at the center of the target zone 146 there would be many positions where it would be unavoidable to irradiate the critical anatomy zone 400. In FIG. 5 the mechanical actuator 104 has moved the location of the rotational point 117 relative to the magnet axis 132. The radiotherapy source 410′ and the beam collimator 412′ have been rotated into a second position for irradiating the target zone 146. The radiation beam 414′ passes through the target zone 146 and avoids the critical anatomy zone 400. The mechanical actuator 104 can therefore be used to effectively avoid irradiating the critical anatomy zone 400.

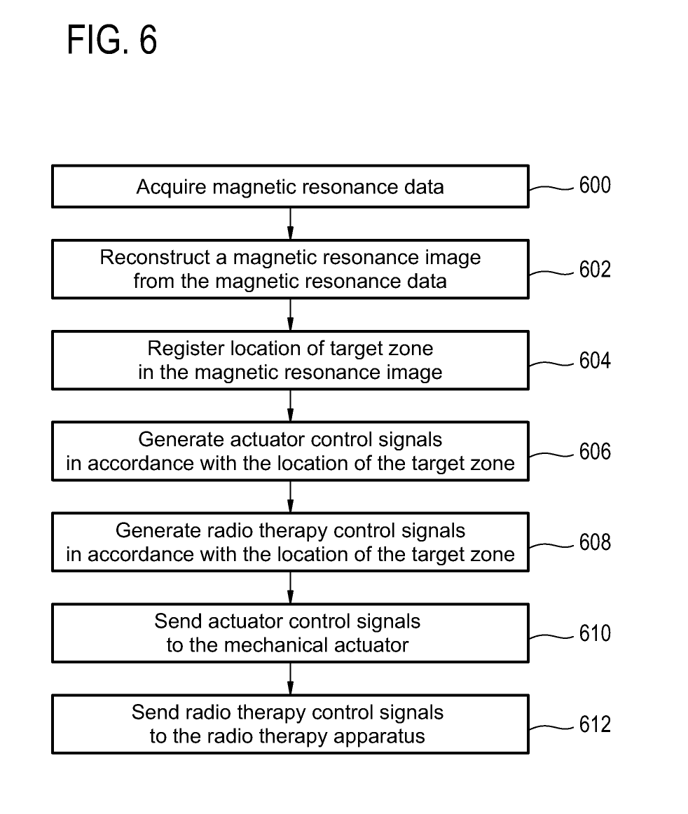

FIG. 6 shows an embodiment of a method according to the invention. The method may be implemented as a computer program product or instructions on a computer-readable storage medium. Alternatively, the method may be implemented as a computer-implemented method also. In step 600 magnetic resonance data is acquired. In step 602 a magnetic resonance image is reconstructed from the magnetic resonance data. In step 604 the location of the target zone is registered in the magnetic resonance image. In step 606 actuator control signals are generated in accordance with the location of the target zone. In step 608 the radiotherapy control signals are generated in accordance with the location of the target zone also. The actuator control signals and the radiotherapy control signals are generated in accordance with each other so that the target zone is effectively treated. In step 610 actuator control signals are sent to the mechanical actuator. In step 612 radiotherapy control signals are sent to the radiotherapy apparatus.

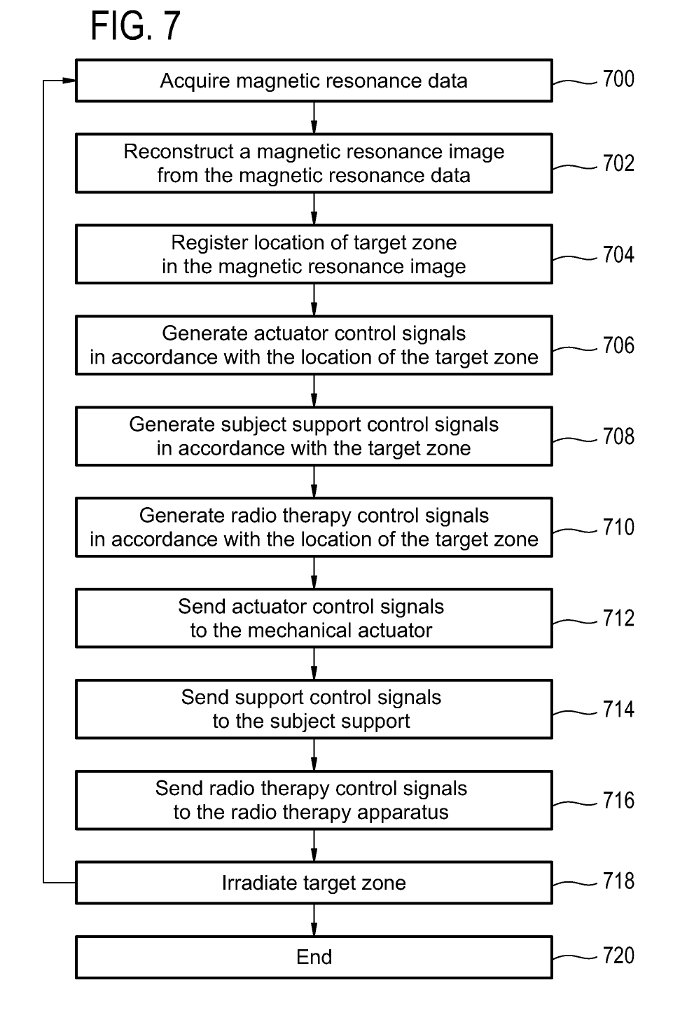

FIG. 7 shows a flow diagram which illustrates a further embodiment of a method according to the invention. As with FIG. 6 the method shown in FIG. 7 may be implemented as a computer program product, as instructions on a computer-readable storage medium, as a computer-implemented method or as a software product. In step 700 magnetic resonance data is acquired. Next in step 702 a magnetic resonance image is reconstructed from the magnetic resonance data. In step 704 the location of the target zone is registered in the magnetic resonance image. In step 706 actuator control signals are generated in accordance with the location of the target zone. In step 708 subject support control signals are generated in accordance with the target zone. In step 710 radiotherapy control signals are generated in accordance with the location of the target zone. The actuator control signals, the subject support control signals and the radiotherapy control signals are all generated in accordance with each other. In step 712 actuator control signals are sent to the mechanical actuator. In step 714 support control signals are sent to the subject support. In step 716 radiotherapy control signals are sent to the radiotherapy apparatus. In step 718 the target zone of the subject is irradiated. During the irradiation the method may loop back to 700 and new magnetic resonance data may be acquired. The process may be repeated continuously during the irradiation to monitor to see if the location of the target zone changes. If the location of the target zone changes new control signals can be generated to compensate for motion of the target zone. After completion of the irradiation, the method ends at step 720.

While the invention has been illustrated and described in detail in the drawings and foregoing description, such illustration and description are to be considered illustrative or exemplary and not restrictive; the invention is not limited to the disclosed embodiments.

Other variations to the disclosed embodiments can be understood and effected by those skilled in the art in practicing the claimed invention, from a study of the drawings, the disclosure, and the appended claims. In the claims, the word “comprising” does not exclude other elements or steps, and the indefinite article “a” or “an” does not exclude a plurality. A single processor or other unit may fulfill the functions of several items recited in the claims. The mere fact that certain measures are recited in mutually different dependent claims does not indicate that a combination of these measured cannot be used to advantage. A computer program may be stored/distributed on a suitable medium, such as an optical storage medium or a solid-state medium supplied together with or as part of other hardware, but may also be distributed in other forms, such as via the Internet or other wired or wireless telecommunication systems. Any reference signs in the claims should not be construed as limiting the scope.

### LIST OF REFERENCE NUMERALS

100 therapeutic apparatus
102 radio therapy apparatus
104 mechanical actuator
106 magnetic resonance imaging system
108 ring mechanism
110 radio therapy source
112 beam collimator
114 radiation beam
116 rotational axis
117 rotational point
118 tilt apparatus
120 direction of tilt
122 magnet
124 cryostat
126 superconducting coil
128 compensation coil
130 low magnetic field zone
132 magnet axis
134 magnetic field gradient coil
136 magnetic field gradient coil power supply
138 imaging zone
140 radio frequency coil
142 radio frequency transceiver
144 subject
146 target zone
148 subject support
150 mechanical positioning system
152 computer system
154 hardware interface
156 processor
158 user interface
160 computer storage
162 computer memory
164 top distance
166 bottom distance
168 treatment plan
170 magnet resonance data
172 magnetic resonance image
174 coordinates of target zone
176 actuator control signals
178 radio therapy control signals
180 therapeutic apparatus control module
182 radio therapy apparatus control module
184 mechanical actuator control module
186 magnetic resonance imaging control module
188 image reconstruction module
190 image registration module
192 actuator control signal generation module
194 radio therapy control signal generation module
200 x-axis
202 y-axis
210 radio therapy source
210′ radio therapy source
212 beam collimator
212′ beam collimator
214 radiation beam
214′ radiation beam
400 critical anatomy zone
410 radio therapy source
410′ radio therapy source
412 beam collimator
412′ beam collimator
414 radiation beam
414′ radiation beam

1. A therapeutic apparatus comprising:
a radio therapy apparatus for treating a target zone of a subject, wherein the radio therapy apparatus comprises a radio therapy source for generating electromagnetic radiation, wherein the radio therapy apparatus is adapted for rotating the radio therapy source about a rotational point;
a mechanical actuator for supporting the radio therapy apparatus and for moving the position and/or orientation of the rotational point; and
a magnetic resonance imaging system for acquiring magnetic resonance data from an imaging zone, wherein the target zone is within the imaging zone, wherein the magnetic resonance imaging system comprises a magnet for generating a magnetic field within the imaging zone, wherein the radio therapy source is adapted for rotating at least partially about the magnet.

2. The therapeutic apparatus of claim 1, wherein the therapeutic apparatus further comprises a processor for controlling the therapeutic apparatus; wherein the therapeutic apparatus further comprises a memory containing machine executable instructions for execution by the processor; wherein execution of the instructions causes the processor to:
acquire the magnetic resonance data using the magnetic resonance imaging system;
reconstruct a magnetic resonance image from the magnetic resonance data;
register a location of the target zone in the magnetic resonance image; and
generate actuator control signals in accordance with the location of the target zone, wherein actuator control signals cause the mechanical actuator to move the position and/or orientation of the rotational point;
generate radio therapy control signals in accordance with the location of the target zone, wherein the radio therapy control signals that cause the radio therapy apparatus to irradiate the target zone and cause the radio therapy apparatus to control rotation of the radio therapy source about the rotational point;
send the actuator control signals to the mechanical actuator; and
send the radio therapy control signals to the radio therapy apparatus.

3. The therapeutic system of claim 2, wherein execution of the instructions causes the processor to generate actuator control signals that cause the mechanical actuator to move such that the rotational point is within a predetermined distance from the target zone.

4. The therapeutic apparatus of claim 2, wherein execution of the instructions further cause the apparatus to register a location of a critical anatomy zone in the magnetic resonance image, and wherein actuator control signals are generated in accordance with the location of the target zone and the critical anatomy zone such that the radiation dose to the critical anatomy zone is minimized and that the radiation dose to the target zone is maximized.

5. The therapeutic apparatus of claim 2, wherein the therapeutic apparatus further comprises a subject support control interface for controlling a subject support for positioning the subject, wherein execution of the instructions further causes the processor to generate subject support control signals, wherein execution of the instructions further cause the processor to send the subject support control signals to the subject support using the subject support interface, wherein the subject support control signals are generated in accordance with the radio therapy control signals and the location of the target zone.

6. The therapeutic apparatus of claim 2, wherein execution of the instructions further cause the processor to:
repeatedly acquire the magnetic resonance data, reconstruct the magnetic resonance image, and register the location of the target zone during irradiation of the target zone; and
repeatedly generate and send updated radio therapy control signals, wherein the updated radio therapy control signals compensate for motion of the subject between subsequent acquisitions of the magnetic resonance data; and wherein the updated radio therapy control signals are sent to the radio therapy source during irradiation of the target zone.

7. The therapeutic apparatus of claim 6, wherein radio therapy apparatus comprises an adjustable beam collimator, and wherein the updated radio therapy control signals comprises commands for controlling the beam collimator.

8. The therapeutic apparatus of, wherein the radio therapy source is adapted for generating a radiation beam with a beam path, wherein the radio therapy apparatus rotates the radio therapy source within a rotational plane wherein the radio therapy apparatus further comprises a tilt apparatus adapted for tilting the beam path relative to the rotational plane.

9. The therapeutic apparatus of claim 8, wherein execution of the instructions further causes the processor to generate tilt apparatus control signals in accordance with the location of the target zone, wherein tilt apparatus control signals cause the tilt apparatus tilt the beam path relative to the rotational plane, and wherein the radio therapy control signals comprise the tilt apparatus control signals.

10. The therapeutic apparatus of claim 1, wherein the radio therapy source is an LINAC for generating X-ray radiation, wherein the magnet is adapted for generating a low magnetic field zone which encircles the magnet, wherein the radio therapy apparatus is adapted such that the radio therapy source rotates about the magnet within the low magnetic field zone, wherein the magnetic field strength within the low magnetic field zone is below a operational threshold of the LINAC source, and wherein the operational threshold defines a magnetic field strength which prevents the LINAC source from functioning.

11. The therapeutic apparatus of claim 10, wherein the operational threshold is below 50 gauss, preferably below 10 gauss.

12. The therapeutic apparatus of claim 1, wherein the radio therapy source is any one of the following: LINAC X-ray source, and X-ray tube, and a radio isotope gamma radiation source.

13. The therapeutic apparatus of claim 1, wherein the mechanical actuator comprises a hydraulic system.

14. A computer program product comprising machine executable instructions for execution by a processor of a therapeutic apparatus, wherein the therapeutic apparatus comprises a radio therapy apparatus for treating a target zone of a subject, wherein the radio therapy apparatus comprises a radio therapy source for generating electromagnetic radiation, wherein the radio therapy apparatus is adapted for rotating the radio therapy source about a rotational point, wherein the therapeutic apparatus further comprises a mechanical actuator for supporting the radio therapy apparatus and for moving the position and/or orientation of the rotational point, wherein the therapeutic apparatus further comprises a magnetic resonance imaging system for acquiring magnetic resonance data from an imaging zone, wherein the target zone is within the imaging zone, wherein the magnetic resonance imaging system comprises a magnet for generating a magnetic field within the imaging zone, wherein the radio therapy source is adapted for rotating at least partially about the magnet, and wherein execution of the instructions causes the processor to:
acquire the magnetic resonance data using the magnetic resonance imaging system;
reconstruct a magnetic resonance image from the magnetic resonance data;
register a location of the target zone in the magnetic resonance image; and
generate actuator control signals in accordance with the location of the target zone, wherein actuator control signals cause the mechanical actuator to move the position and/or orientation of the rotational point;
generate radio therapy control signals in accordance with the location of the target zone, wherein the radio therapy control signals cause the radio therapy apparatus to irradiate the target zone and cause the radio therapy apparatus to control rotation of the radio therapy source about the rotational point;
send the actuator control signals to the mechanical actuator; and
send the radio therapy control signals to the radio therapy apparatus.

15. A computer-implemented method of controlling a therapeutic apparatus, wherein the therapeutic apparatus comprises a radio therapy apparatus for treating a target zone, of a subject, wherein the radio therapy apparatus comprises a radio therapy source for generating electromagnetic radiation, wherein the radio therapy apparatus is adapted for rotating the radio therapy source about a rotational point, wherein the therapeutic apparatus further comprises a mechanical actuator for supporting the radio therapy apparatus and for moving the position and/or orientation of the rotational point, wherein the therapeutic apparatus further comprises a magnetic resonance imaging system for acquiring magnetic resonance data from an imaging zone, wherein the target zone is within the imaging zone, wherein the magnetic resonance imaging system comprises a magnet for generating a magnetic field within the imaging zone, wherein the radio therapy source is adapted for rotating at least partially about the magnet, and the method comprises the steps of:
acquiring the magnetic resonance data using the magnetic resonance imaging system;
reconstructing a magnetic resonance image from the magnetic resonance data;
registering a location of the target zone in the magnetic resonance image; and
generating actuator control signals in accordance with the location of the target zone, wherein the actuator control signals cause the mechanical actuator to move the position of and/or orientation of the rotational point;
generating radio therapy control signals in accordance with the location of the target zone, wherein the radio therapy control signals cause the radio therapy apparatus to irradiate the target zone and cause the radio therapy apparatus to control rotation of the radio therapy source about the rotational point;
sending the actuator control signals to the mechanical actuator; and
sending the radio therapy control signals to the radio therapy apparatus.
